(19) United States
(12) Patent Application Publication
(10) Pub. No.: US 2009/0124966 A1
(43) Pub. Date: May 14, 2009

(54) AMPOULE USABLE AS A SYRINGE AND A SYRINGE UNIT COMPRISING THE AMPOULE

(76) Inventor: Yoshio Oyama, Tokyo (JP)

Correspondence Address: MYERS WOLIN, LLC, 100 HEADQUARTERS PLAZA, North Tower, 6th Floor, MORRISTOWN, NJ, 07960-6834, US

(21) Appl. No.: 12/161,843

(22) PCT Filed: Jan. 13, 2007
(86) PCT No.: PCT/JP2007/000011

§ 371 Date: Jan. 20, 2009

(30) Foreign Application Priority Data
Jan. 23, 2006 (JP) 2006-013876
May 23, 2006 (JP) 2006-142306

(51) Int. Cl. A61M 5/30

(52) U.S. Cl. 604/68

(57) ABSTRACT

The object is to provide an ampoule usable as a syringe, and a syringe unit which are easy to handle, excellent in quantitative accuracy, and further more excellent in hygienic aspect.

The above object is solved by an ampoule usable as a syringe comprising: a front edge portion (4) which is removed when the ampoule is used; an ampoule body (2) which can accommodate a drug solution (10); and a liquid infusing portion (3) connecting the front edge portion (4) and the ampoule body (2); the ampoule body (2) being able to place a movable stopper (5) in contact with an internal surface of the ampoule body (2), and being able to accommodate the drug solution (10) hermetically-sealed in the through-hole (6) between the movable stopper (5) and the sealing portion (4a).

### FIELD OF THE INVENTION

The present invention relates to an ampoule usable as a syringe for administering drug solution to a dynamic body of a patient, an animal, or the like. Also, the present invention relates to a syringe unit provided with an ampoule usable as a syringe and an ampoule holder.

### BACKGROUND ART

Generally, an injection is performed by a needle-tipped syringe. However, using a syringe needle gives fear and ache to the patient to be medicated. Specifically, in medical treatment or diagnosis for children, many children fear the syringe needle, which results in an obstacle of the medical treatment or the like. Also, the handling of the syringe needle after use for a patient requires considerable care. If mishandled, there is a high risk of infection to other patients or medical staffs. Therefore, used syringe needles are disposed as a medical waste.

Therefore, needleless syringes with no needles have been developed and gradually used in recent years as substitute for medication using needles. A needleless syringe shoots the drug solution at an extremely high speed towards the skin. Then, a hole which is smaller than that of a needle of the syringe was made at the skin, so that the drug solution penetrates through the skin. Accordingly, the drug solution can be administered within the skin (see for example, patent document 1). Such a medication using the needleless syringe does not give any pain to the patient since no needle is used, and is free of infectious risk due to used syringe needles. Also, since the drug solution directly penetrates the skin, the drug solution diffuses within the skin. As a result, the infiltration of the drug solution is faster than the case where the needle is used. Therefore, a time before an effect of the medication is seen can be shortened compared to the case where the needle is used.

Specifically, there are many occasions recently where injection for medication is personally performed. For example, in insulin administration for diabetical medication, heparin administration for thrombotic disease prevention, alpha interferon administration as cancer treatment for recuperation at home, and the like, the patient himself or herself is daily required to administer the drug solution into the body by the injection. Therefore, in the presence of pain due to a needle insertion, the patient may become hesitant to continue the prevention or medication, so that the needleless syringe with no needle has been used.

However, the above-mentioned needleless syringe has a complicated composition, where the drug solution is measured and suctioned to be shot upon every use. Therefore the handling thereof is complicated. Also, in the course of repeating the task of measuring the drug solution to be suctioned by the needleless syringe, the amount of the drug solution suctioned by the needleless syringe may vary, resulting in a lack of accuracy of the suctioned amount of the drug solution. Moreover, since the same needleless syringe is used again and again for suctioning the drug solution for administration, problems exist from a hygienic perspective.

For example, in case of a conventional needleless syringe (30) shown in FIG. 13, a connecting portion (34) of the needleless syringe (30) is fitted in a vial container that is not shown to measure the drug solution by pulling a piston (33) in accordance with the scale indicated on an ampoule body (32). Therefore, the handling thereof is inconvenient and errors of the measured amount of drug solution are easy to occur. Namely, since the needleless syringe (30) is filled with the drug solution from the vial container including a large amount of drug solution for use of several tens to several hundreds of injections, the connecting portion (34) is structurally required for connection between the vial container and the needleless syringe (30). Moreover from hygienic perspective, such a connecting portion (34) is required sterilizing. Therefore, the handling of the conventional needleless syringe has been inconvenient. Also, failing to inject an appropriate amount of drug solution causes problems such as excessive intake and side-effects, so that errors in the amount of measured drug solution may cause risk for the administered dynamic body.

Also for the needleless syringe (30), it is preferable to use a strong material tolerant of shock upon injection. For this reason, the needleless syringe (30) inevitably uses an expensive material. Also for this reason, the used needleless syringe (30) is often reused, thereby causing hygienical issues.

### REFERENCES

Patent document 1: Japanese Patent Application Laid-Open Publication No.

### DISCLOSURE OF THE INVENTION

### Problems to be Solved by the Invention

An object of the present invention is to provide an ampoule usable as a syringe and an ampoule unit, which are easy to handle, have excellent quantitative accuracy, and are hygienically excellent.

### Means for Solving the Problems

The present invention is based on a knowledge that basically an ampoule accommodating a fixed amount of a drug solution can be obtained by enabling a movable stopper to be inserted from lower side of a body of the ampoule and providing the ampoule with a front edge portion which is removed upon use, and that by pushing out the movable stopper in a state where the front edge portion is removed, the ampoule can be used as a syringe.

### Means for Solving the Problems

Namely, the first aspect of the present invention is related to an ampoule usable as a syringe comprising: a front edge portion (4) which is removed when the ampoule is used; an ampoule body (2) which can accommodate a drug solution (10); and a liquid infusing portion (3) connecting the front edge portion (4) and the ampoule body (2); wherein the front edge portion (4), the ampoule body (2), and the liquid infusing portion (3) being integrally formed; a through-hole (6) being formed in the front edge portion (4), the ampoule body (2), and the liquid infusing portion (3) penetrating from the ampoule body (2) to the midway through the front edge portion (4); the front edge portion (4) having a sealing portion (4a) sealing the through-hole (6); and the ampoule body (2) being able to place a movable stopper (5) in contact with an internal surface of the ampoule body (2), and being able to accommodate the drug solution (10) hermetically-sealed in the through-hole (6) between the movable stopper (5) and the sealing portion (4a).

Since the ampoule (1) according to the first aspect of the present invention can be previously filled with the drug solution (10) by being sealed with the movable stopper (5), measuring the drug solution (10) upon every use thereof becomes no longer necessary. As a result, with the ampoule (1) according to the first aspect of the present invention, the handling is made easy and a measurement mistake of the injected quantity can be prevented. Also, the drug solution (10) hermetically sealed in the ampoule (1) is opened and used up for every use, so that the ampoule (1) according to the first aspect of the present invention is hygienically favorable, and a risk of infection by reusing the ampoule (1) is small. Moreover, if the ampoule (1) is made of resin, a solution processing and a thermal disposal can be performed as is after use, so that the disposal after use is easy.

Specifically, for a person requiring a daily intake of a medical agent by an injection at home, it is important that the syringe is easy to handle and is capable of administering an accurate amount of the medical agent. Since the ampoule (1) according to the first aspect of the present invention can be used as a disposable type syringe filled with an accurate amount of the medical agent, there is no need for the above-mentioned person requiring a daily intake of a medical agent by a daily injection at home to measure the drug solution (10) by himself or herself, there is no measurement mistake of the injected amount, and the handling is easy.

A preferred embodiment of the first aspect of the present invention is related to the above-mentioned ampoule (1) usable as a syringe, wherein a liquid infusing hole (7), that is a portion of the through-hole (6) and formed in the liquid infusing portion (3), has a diameter decreased in the direction from the ampoule body (2) to the front edge portion (4).

Instead of having a needle, it is preferable that the needleless syringe has a bore diameter at the time of injection made small in order to increase an injection pressure of the drug solution (10). If the liquid infusing hole (7) is made to have a small diameter entirely from the side of the ampoule body (2), the loss of pressure having pushed out from the ampoule body (2) to the liquid infusing hole (7) becomes large. On the other hand, if the liquid infusing hole (7) is made large in order to suppress this loss of pressure, the bore diameter at the time of injection also becomes large, so that the drug solution (10) is injected to the dynamic body at a certain level of injection pressure, which may possibly increase the pain.

In this regard, the liquid infusing hole (7) of the ampoule (1) according to this embodiment has the diameter gradually decreased in the direction from the ampoule body (2) to the side of the front edge portion (4). Therefore, the loss of injection pressure of the drug solution (10) upon injection of the drug solution (10) can be reduced and the bore diameter at the time of injection can be made small.

A preferred embodiment of the first aspect of the present invention is related to the any of the above-mentioned ampoule (1) usable as a syringe, wherein a taper portion (3a) tapering off from the liquid infusing portion (3) to a side of the front edge portion (4) is formed at a tip of the liquid infusing portion (3) on the side of the front edge portion (4).

According to the ampoule (1) of this embodiment, the front edge of the liquid infusing portion (3) can be contacted to the skin more closely by the taper portion (3a). Therefore, the drug solution (10) is enabled to reach within the skin more reliably without causing dripping at the time of the needleless injection.

A preferred embodiment of the first aspect of the present invention is related to any of the above-mentioned ampoule (1) usable as a syringe, wherein a taper portion (3a) tapering off from the liquid infusing portion (3) to a side of the front edge portion (4) is formed at a tip of the liquid infusing portion (3) on the side of the front edge portion (4), and a contacting portion (3b) having a flat surface is provided around the taper portion (3a).

According to the ampoule (1) of this embodiment, the front edge of the liquid infusing portion (3) can be contacted to the skin more closely by the taper portion (3a) and the contacting portion (3b). Therefore, the drug solution (10) is enabled to reach within the skin more reliably without causing dripping at the time of the needleless injection.

A preferred embodiment of the first aspect of the present invention is related to any of the above-mentioned ampoule (1) usable as a syringe, wherein a portion in the front edge portion (4) connected to the liquid infusing portion (3) is provided with a notched portion (8) for cutting off the front edge portion (4) from the liquid infusing portion (3).

Since the conventional needleless syringe was intended to be used repeatedly, a front edge portion (35) of the liquid infusing hole (see FIG. 13) has always been exposed, so that it was hygienically unfavorable.

According to the ampoule (1) of this embodiment of the present invention, the drug solution (10) can be hermetically sealed therein continuously until immediately before the use of the ampoule (1). Also, by removing the front edge portion (4) immediately before using the ampoule (1) to expose the front edge of the liquid infusing hole (7), it is made possible to perform a drug administration that is hygienically excellent.

A preferred embodiment of the first aspect of the present invention is related to any of the above-mentioned ampoule (1) usable as a syringe, wherein at a bottom face portion (2a) provided on a side of the liquid infusing portion (3) in the internal surface, the ampoule body (2) has a shock absorb means for absorbing an impact of the movable stopper (5).

Generally, while the needleless syringe improves the ache or pain due to a needle insertion as in the conventional needle-tipped syringe, since a piston (33) (see FIG. 13) and the movable stopper are pushed at a high pressure, a considerable impact noise may be generated upon collision of the movable stopper and the bottom face portion of the ampoule body. Such an impact noise may become a cause as a noise replacing the ache or pain for a patient to hesitate the use of the needleless syringe.

In this regard, according to the ampoule (1) of this embodiment, the impact noise can be reduced by the shock absorb means for absorbing the impact, so that it is made possible to reduce the noise (impact noise) generated by the impact on the bottom face portion (2a). Thus, it is made possible to perform a drug administration free of unpleasantness and aversion.

A preferred embodiment of the first aspect of the present invention is related to any of the above-mentioned ampoule (1) usable as a syringe, wherein a spiral concavity and convexity are formed on an external surface of the ampoule body (2).

According to the ampoule (1) of this embodiment of the present invention, by forming a spiral concavity and convexity on an external surface of the ampoule body (2), it is made possible to increase the strength of the ampoule body (2). With the ampoule (1) of this embodiment, use of a holder for accommodating the ampoule (1) becomes unnecessary since the strength is excellent. As a result, the ampoule (1) can be handled easily.

Moreover, since the concavity and the convexity formed on the external surface of the ampoule body (2) are spiral, the ampoule (1) of this embodiment can be manufactured by a rotational press fitting into a molding die. At this time, the spiral concavity and convexity is formed simultaneously with the press fitting in association with the rotation, so that it is made possible to improve the productive efficiency of the ampoule (1).

A preferred embodiment of the first aspect of the present invention is related to any of the above-mentioned ampoule (1) usable as a syringe, wherein a plurality of annular concavities and convexities are formed on an external surface of the ampoule body (2).

According to the ampoule (1) of this embodiment of the present invention, by forming annular concavities and convexities on an external surface of the ampoule body (2), it is made possible to increase the strength of the ampoule body (2). With the ampoule (1) of this embodiment, use of a holder for accommodating the ampoule (1) becomes unnecessary since the strength is excellent. As a result, the ampoule (1) can be handled easily.

Moreover, since the concavities and the convexities formed on the external surface of the ampoule body (2) are annular, the ampoule (1) of this embodiment can be manufactured by rotating a molding die. By rotating the molding die, it is made possible to easily manufacture the product without irregularity in thickness, so that it is made possible to improve the productive efficiency of the ampoule (1).

A preferred embodiment of the first aspect of the present invention is related to any of the above-mentioned ampoule (1) usable as a syringe, wherein the liquid infusing portion (3) after having the front edge portion (4) removed therefrom has a shape which enables a syringe needle to be attached thereto.

Namely, the ampoule (1) of the present invention can be basically used for a needleless syringe. However, use as a needle-tipped syringe may be possible by attaching a needle as in the ampoule (1) of this embodiment.

A preferred embodiment of the first aspect of the present invention is related to any of the above-mentioned ampoule (1) usable as a syringe, further comprising a movable stopper (5) in a hollow portion of the ampoule body (2).

Since the ampoule (1) of this embodiment is provided with a movable stopper (5), the drug solution (10) can be hermetically sealed therein.

A preferred embodiment of the first aspect of the present invention is related to any of the above-mentioned ampoule (1) usable as a syringe, further comprising a movable stopper (5) in a hollow portion of the ampoule body (2), wherein a concavity and a convexity contacting with an internal surface of the ampoule body (2) are formed on a side face of the movable stopper (5).

The side face of the movable stopper (5) means, for example, a periphery of the movable stopper (5) facing the internal surface of the ampoule body (2) in a direction perpendicular to a central axis of the ampoule body (2) when the movable stopper (5) is placed in a hollow portion of the ampoule body (2).

According to ampoule (1) of this embodiment, the airtightness with the ampoule body (2) is increased by the movable stopper (5), so that the drug solution (10) can be hermetically-sealed reliably, and the friction between the movable stopper (5) and the internal surface of the ampoule body (2) is reduced by the concavity and the convexity of the movable stopper, thereby suppressing the speed reduction of the movable stopper (5) due to the injection by the needleless injection compared to the case where the movable stopper (5) doesn't have the above-mentioned concavity and convexity. Thus, the decrease in injection pressure of the drug solution (10) can be suppressed.

A preferred embodiment of the first aspect of the present invention is related to any of the above-mentioned ampoule (1) usable as a syringe, further comprising a movable stopper (5) in a hollow portion of the ampoule body (2), wherein a drug solution (10) is accommodated in the through-hole (6) hermetically sealed by the movable stopper (5).

Since the ampoule (1) of this embodiment has the drug solution (10) hermetically sealed therein, an accurate amount of the drug solution (19) can be administered by using the ampoule (1) of this embodiment.

The second aspect of the present invention is related to a syringe unit comprising: an ampoule (1); a movable stopper (5) accommodated in the ampoule (1); and a holder (20) accommodating the ampoule (1); wherein the ampoule (1) comprises: a front edge portion (4) which is removed upon use; an ampoule body (2) filled with a drug solution (10); and a liquid infusing portion (3) connecting the front edge portion (4) and the ampoule body (2); the front edge portion (4), the ampoule body (2), and the liquid infusing portion (3) being integrally formed, a through-hole (6) being formed in the front edge portion (4), the ampoule body (2), and the liquid infusing portion (3) penetrating from the ampoule body (2) to the midway through the front edge portion (4); the front edge portion (4) having a sealing portion (4a) sealing the through-hole (6), and the ampoule body (2) being able to place a movable stopper (5) in contact with an internal surface of the ampoule body (2), and being able to be filled with the drug solution (10) hermetically-sealed in the through-hole (6) between the movable stopper (5) and the sealing portion (4a).

In the second aspect of the present invention, any of the above-mentioned ampoule (1) may be used.

A preferred embodiment of the second aspect of the present invention is related to the above-mentioned syringe unit, wherein the ampoule (1) and the holder (20) are integrally formed.

Since the ampoule (1) and the holder (20) are integrally formed in the syringe unit of this embodiment, a task of inserting the ampoule (1) usable as a syringe into the holder (20) becomes unnecessary. Therefore, compared to the case where the ampoule (1) and the holder (20) are not integrally formed, the handling becomes easier. By using the syringe unit of this embodiment, not only the handling is made easier but also a drug administration which is excellent in quantitative accuracy and in hygienic aspect is made possible.

### EFFECTS OF THE INVENTION

As described above, an ampoule usable as a syringe and an ampoule unit of the present invention are easy to handle and are excellent in quantitative accuracy and are further excellent in hygienic aspect.

### BRIEF DESCRIPTION OF THE DRAWINGS

FIG. 1 is a diagram showing an ampoule (1) usable as a syringe according to the first embodiment of the present invention. FIG. 1(a) is a side view of the ampoule (1) usable as a syringe, FIG. 1(b) is a cross-sectional view of the ampoule (1), and FIG. 1(c) is an enlarged view of a portion X in FIG. 1(b).

FIG. 2 is a diagram showing an example of forming a liquid infusing hole (7). FIGS. 2(a)-(f) respectively show different formation examples where the diagrams on the left are enlarged side views and the diagrams on the right are enlarged elevation views.

FIG. 3 is a diagram showing a holder of an ampoule usable as a syringe.

FIG. 4 is a diagram showing a state where a needleless syringe ampoule is inserted into a holder (20).

FIG. 5 is a diagram showing a syringe unit having a holder and an ampoule usable as a syringe integrally formed.

FIG. 6 is a diagram showing a state where a needleless syringe unit according to the first embodiment of the present invention is used. FIG. 6(a) is a diagram of early phase of a use of a needleless syringe unit, FIG. 6(b) is a diagram of an interim phase of a use of a needleless syringe unit, and FIG. 6(c) is a diagram of a late phase of a use of a needleless syringe unit.

FIG. 7 is a diagram showing a usage state where a syringe needle is used for a syringe unit according to the first embodiment of the present invention.

FIG. 8 is a process explanatory diagram for describing a manufacturing process of an ampoule usable as a syringe according to the present invention. FIG. 8(a) is a diagram of an early manufacturing phase of an ampoule, FIG. 8(b) is a diagram showing an early interim manufacturing phase of an ampoule, FIG. 8(c) is a diagram showing a late interim manufacturing phase of an ampoule, FIG. 8(d) a diagram showing a late manufacturing phase of an ampoule, FIG. 8(e) is an enlarged view of a portion Y in FIG. 8(d), and FIG. 8(f) is a diagram showing a completed state of an ampoule (1).

FIG. 9 is a diagram showing an ampoule usable as a syringe according to the second embodiment of the present invention. FIG. 9(a) is a side view of the ampoule, FIG. 1(b) is a cross-sectional view of the ampoule, FIG. 1(c) is an enlarged view of a portion X in FIG. 9(b), and FIG. 9(d) is an enlarged view of a portion Y in FIG. 9(c).

FIG. 10 is a side view of an ampoule usable as a syringe according to the third embodiment of the present invention.

FIG. 11 is a diagram showing a state where a needleless syringe unit according to the second (and the third) embodiment of the present invention is used. FIG. 11(a) is a diagram of early phase of a use of a needleless syringe unit, FIG. 11(b) is a diagram of an interim phase of a use of a needleless syringe unit, and FIG. 11(c) is a diagram of a late phase of a use of a needleless syringe unit.

FIG. 12 is a diagram showing a state where a syringe needle is used for a syringe unit according to the second (and the third) embodiment of the present invention.

FIG. 13 is a diagram showing a conventional needleless syringe.

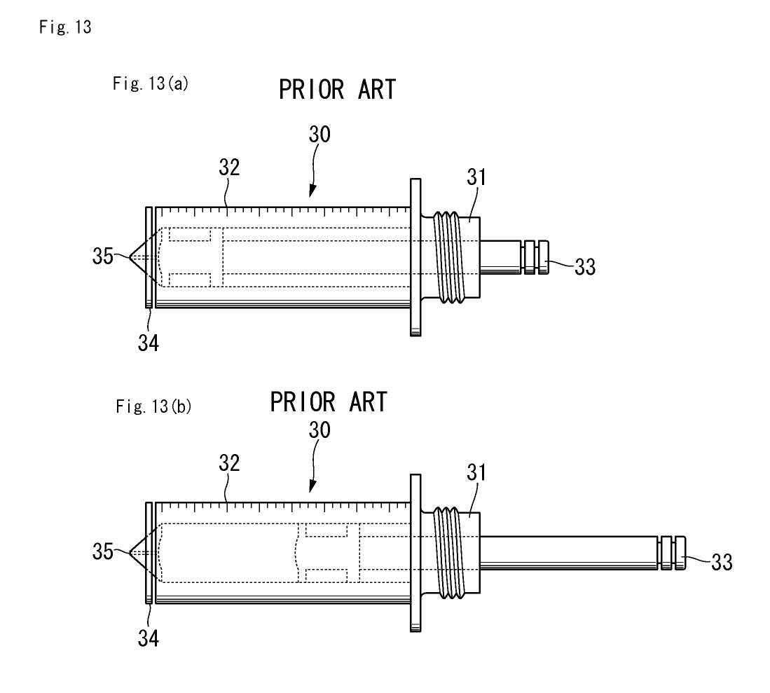

### DESCRIPTION OF THE ELEMENT NUMERALS

1 ampoule usable as a syringe
2 ampoule body
3 liquid infusing portion
3a taper portion
3b contacting portion
4 front edge portion
5 movable stopper
6 through-hole
7 liquid infusing hole
8 notched portion
10 drug solution
20 holder
22 syringe needle connecting portion
23 syringe needle
30 needleless syringe (prior art)
40 main-molding dies
41 sub-molding die

### The Best Mode of the Invention

Hereinafter, best mode for carrying out the present invention will be described based on the drawings.

### I FIRST EMBODIMENT

### 1. Ampoule Usable as Syringe

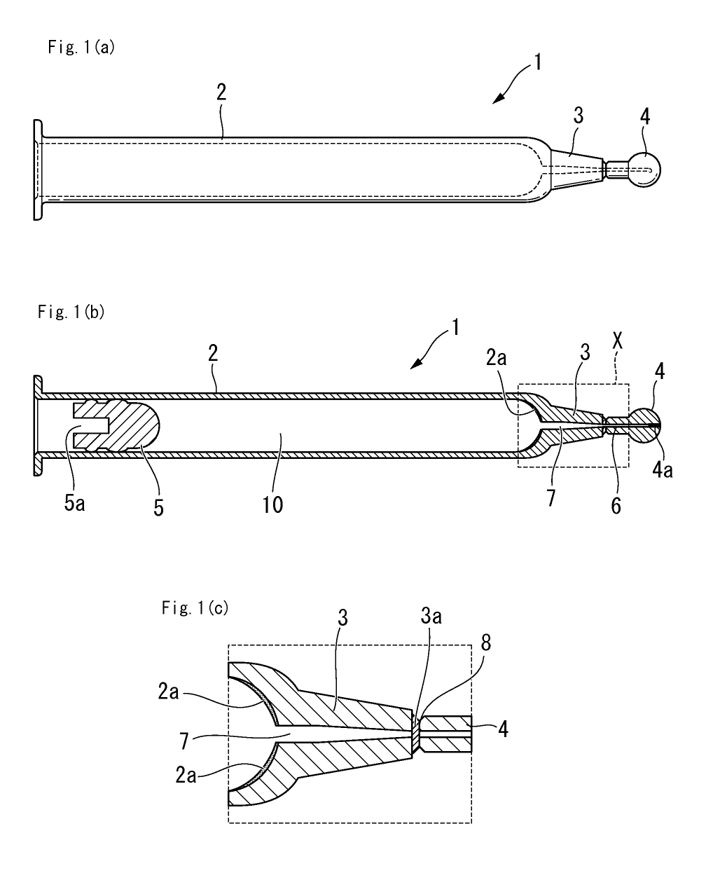

FIG. 1 is a diagram showing an ampoule (1) usable as a syringe according to the first embodiment of the present invention. FIG. 1 (a) is a side view of the ampoule (1) usable as a syringe (also referred to as a syringe ampoule), FIG. 1(b) is a cross-sectional view thereof, and FIG. 1(c) is an enlarged view of a portion X in FIG. 1(b). Hereinafter, taking a needleless syringe as an example of the ampoule (1) usable as a syringe, the needleless syringe ampoule, an ampoule body (2), a liquid infusing portion (3), a front edge portion (4), and the like will be described.

In FIG. 1, the ampoule (1) usable as a syringe according to the first embodiment of the present invention is composed of a front edge portion (4) which is removed upon use; an ampoule body (2) which can accommodate a drug solution (10); and a liquid infusing portion (3) connecting the front edge portion (4) and the ampoule body (2); the front edge portion (4), the ampoule body (2), and the liquid infusing portion (3) are integrally formed; a through-hole (6) is formed in the front edge portion (4), the ampoule body (2), and the liquid infusing portion (3) penetrating from the ampoule body (2) to the midway through the front edge portion (4); the front edge portion (4) has a sealing portion (4a) sealing the through-hole (6); and the ampoule body (2) is able to place a movable stopper (5) in contact with an internal surface of the ampoule body (2), and is able to be accommodate with the drug solution (10) hermetically-sealed in the through-hole (6) between the movable stopper (5) and the sealing portion (4a). The ampoule (1) of the present invention is preferably formed of a cyclic olefin copolymer. By forming the ampoule (1) of the present invention with a cyclic olefin copolymer which is excellent in high stiffness and low ductility, the liquid infusing portion (3) and the front edge portion (4) can be cut off easily at the notched portion (8), and the front edge of the liquid infusing portion (3) has its surface smoothly formed, so that the cohesiveness of the front edge of the liquid infusing portion (3) with the skin can be further improved.

The internal surface of the ampoule body (2) is preferably formed smoothly so that the movable stopper (5) can move. Within the ampoule body (2), the drug solution (10) is preferably filled. As for the amount of the drug solution (10), 0.5 cc may be mentioned. Scales like per 0.1 cc may be marked so that a filling amount may be recognized at a glance. Also, as a preferred example of the ampoule body (2), all or a part of the ampoule body (2) is transparent or translucent, so that the color, the sediment, and the like of the drug solution (10) filled can be recognized. By using such an ampoule (1), an abnormality of the drug solution (10) can be found before the use. Moreover, the ampoule body (2) preferably has a shock absorb means at a bottom face portion (2a) provided on the side of the liquid infusing portion (3) in the internal surface, so that the shock absorb means absorbs the impact of the movable stopper (5). Thus, an impact noise between the movable stopper (5) and the bottom face portion (2a) of the ampoule body (2) can be reduced.

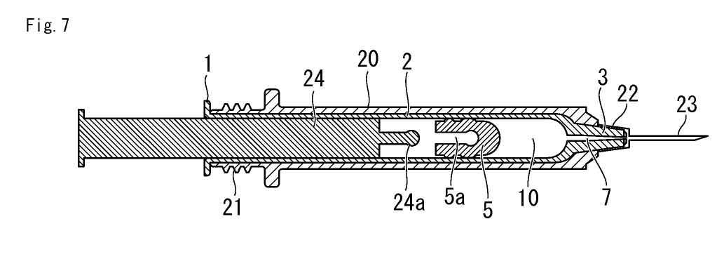

The movable stopper (5) placed in contact with the internal surface of the ampoule body (2) preferably has a concavity and a convexity formed on a surrounding side face thereof. A front edge (head) of the movable stopper (5) is preferably formed a convex curve of the same shape as that of the bottom face (2a) of the ampoule body (2). Also, at a back end of the movable stopper (5), engaging portion for engaging with a plunger (24) or the like shown in FIG. 7 for enabling the move of the movable stopper (5) is formed, and an engaging concave portion (5a) is formed as shown in FIG. 1(b) for example.

By forming the concavity and the convexity on the surrounding side face of the movable stopper (5), the movable stopper (5) contacts with the internal surface of the ampoule body (2), and it is made possible to hermetically seal the drug solution (10) between the movable stopper (5) and the sealing portion (4a). Therefore, the airtightness with the ampoule body (2) is increased by the movable stopper (5), so that the drug solution (10) can be hermetically-sealed reliably, and the friction between the movable stopper (5) and the internal surface of the ampoule body (2) is reduced by the concavity and the convexity of the movable stopper, thereby preventing the situation where the movement speed of the movable stopper (5) is reduced due to the shot by the needleless injection. Thus, the decrease in injection pressure of the drug solution (10) can be prevented.

To describe in more detail, while the concavity and the convexity formed on the surrounding side face of the movable stopper (5) contacts with the internal surface of the ampoule body (2), the contact area of the movable stopper (5) and the internal surface of the ampoule body (2) can be made smaller by the concavity and the convexity compared to the case where the side face of the movable stopper (5) is formed flat. Accordingly, the friction between the movable stopper (5) and the internal surface of the ampoule body (2) can be reduced as described above. Also, while only a single tier of the concavity and the convexity of the movable stopper (5) may provide hermetically-sealed state with the inner ampoule body (2), multiple tiers can steadily maintain the hermetically-sealed state with other tiers of the concavity and the convexity even if a contact of a single tier is weak.

A coaxial through-hole (6) is preferably formed in the liquid infusing portion (3) and the front edge portion (4), and the through-hole (6) is sealed by the sealing portion (4a) at the front edge of the front edge portion (4). In the through-hole (6), a liquid infusing hole (7) formed in the liquid infusing portion (3) preferably has its diameter decreased in the direction from the ampoule body (2) to the front edge portion (4). More specifically, the diameter of the liquid infusing hole (7) on the side of the front edge portion (4) is formed smaller than the diameter of the liquid infusing hole (7) on the side of the ampoule body (2), where the former diameter is formed about 0.15-0.17 mm and the latter diameter is formed about 0.6 mm.

It is to be noted that the shape of the front edge portion (also referred to as “pinch portion”) (4) is not limited to that shown in FIG. 1, and another shape may be adopted. Also, for the sealing portion (4a), any sealing method may be used including a sealing by welding, a sealing by a stopper, and the like as long as the through-hole (6) can be hermetically sealed.

While one liquid infusing hole (7) is formed in the first embodiment of the present invention, a plurality of liquid infusing holes may be formed as the liquid infusing hole (7).

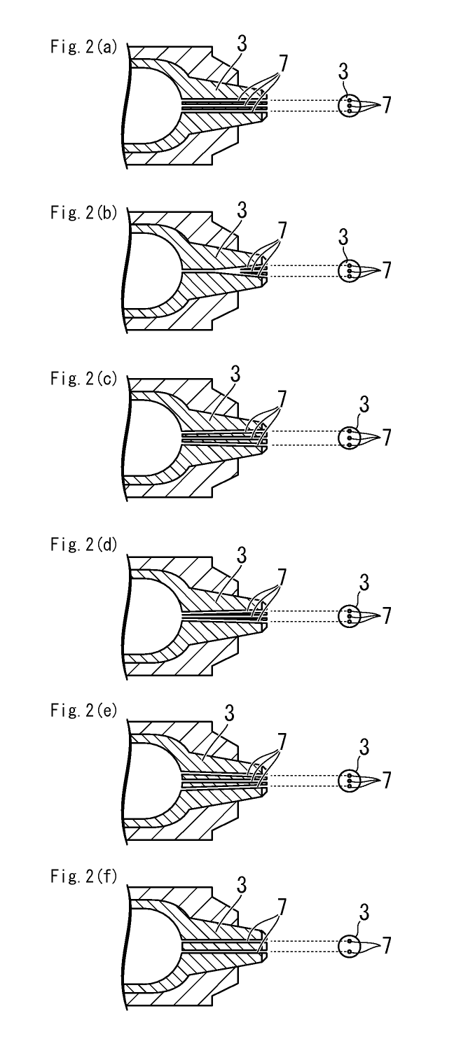

FIG. 2 is a diagram showing formation examples of the liquid infusing hole (7). FIGS. 2 (a)-(f) show enlarged side views (diagrams on the left) and enlarged elevation views (diagrams on the right) of the liquid infusing portion (3). The liquid infusing hole (7) formed in the liquid infusing portion (3) may be formed as two holes, or three or more holes as shown in FIGS. 2(a)-(f) for example. Namely, various configurations of the liquid infusing hole (7) may be formed such as a configuration with three liquid infusing holes 7 parallel in the long direction as shown in FIG. 2(a), a configuration with one first infusing opening located at a border of the ampoule body (2) and the liquid infusing portion (3) and three liquid infusing holes (7) which are radically branched within the liquid infusing portion (3) as shown in FIG. 2(b), a configuration with three first infusing openings located at the border of the ampoule body (2) and the liquid infusing portion (3) and radical three liquid infusing holes (7) as shown in FIG. 2(c), a configuration with one first infusing opening located at the border of the ampoule body (2) and the liquid infusing portion (3) and three liquid infusing holes (7) which are radically branched from the first infusing opening as shown in FIG. 2(d), a configuration with three first infusing openings located at the border of the ampoule body (2) and the liquid infusing portion (3) and three liquid infusing holes (7) which are radically converged as shown in FIG. 2(e), and a configuration with two liquid infusing holes 7 parallel in the long direction as shown in FIG. 2(f).

Also, a notched portion (8) is preferably provided at a part connected to the liquid infusing portion (3) in the front edge portion (4) for disconnecting the front edge portion (4) from the liquid infusing portion (3). This notched portion (8) is, for example, a circular cord (a notch formed by being dented inward along the diameter of the ampoule (1)) for disconnecting the front edge portion (4) from the liquid infusing portion (3). By having such a notched portion (8), it is made possible to easily remove the front edge portion (4) on the boundary of the notched portion (8).

To describe in more detail, when a force is applied to the front edge portion (4) by a twist of the front edge portion (4) or the like, the stress concentrates on the notched portion (8). As a result, the front edge portion (4) and the liquid infusing portion (3) can be easily disconnected on the boundary of the notched portion (8).

For example, the taper portion (3a) is formed about 0.3 mm in height and about 2.0-1.2 mm in width (diameter), thereby increasing the cohesiveness between the tip of the liquid infusing portion (3) and the skin. Also, the drug solution (10) is enabled to reach within the skin more reliably without causing dripping at the time of the needleless injection.

It is to be noted that while the taper portion (3a) is formed in the first embodiment of the present invention, the taper portion (3a) need not be formed as long as the high level of cohesiveness with the skin can be maintained.

2. Syringe unit

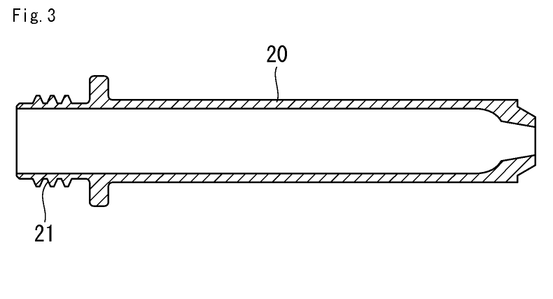

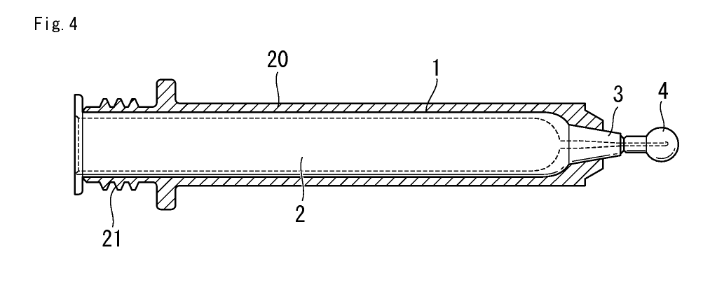

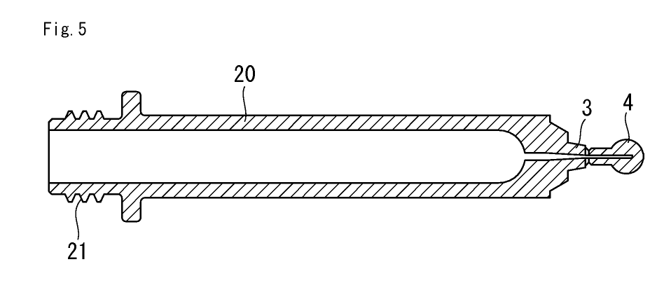

FIG. 3 is a diagram showing a holder (20) of the ampoule (1) usable as a syringe. FIG. 4 is a diagram showing a state where the syringe ampoule (1) is inserted into the holder (20). Also, FIG. 5 is a diagram showing a syringe unit in which the holder (20) and the ampoule (1) usable as a syringe is integrally formed.

In FIG. 3 and FIG. 4, the holder (20) is preferably formed as having approximately the same inner diameter as an outer diameter of the ampoule (1) usable as a syringe, and this holder (20) has a shape where the ampoule (1) usable as a syringe can just fit at a location where the tip of the liquid infusing portion (3) is slightly out from the holder (20). Also, a connecting portion (21) for connecting to a plunger or the like is formed in the holder (20).

When the ampoule (1) usable as a syringe is inserted, the holder (20) accommodates the ampoule body (2) and the liquid infusing portion (3) to hold the ampoule (1).

It is to be noted that the holder (20) and the ampoule (1) usable as a syringe may be integrally formed to obtain the syringe unit as shown in FIG. 5. In this case, the handling becomes easier since a task of inserting the ampoule (1) usable as a syringe into the holder (20) becomes unnecessary.

### 3. Usage

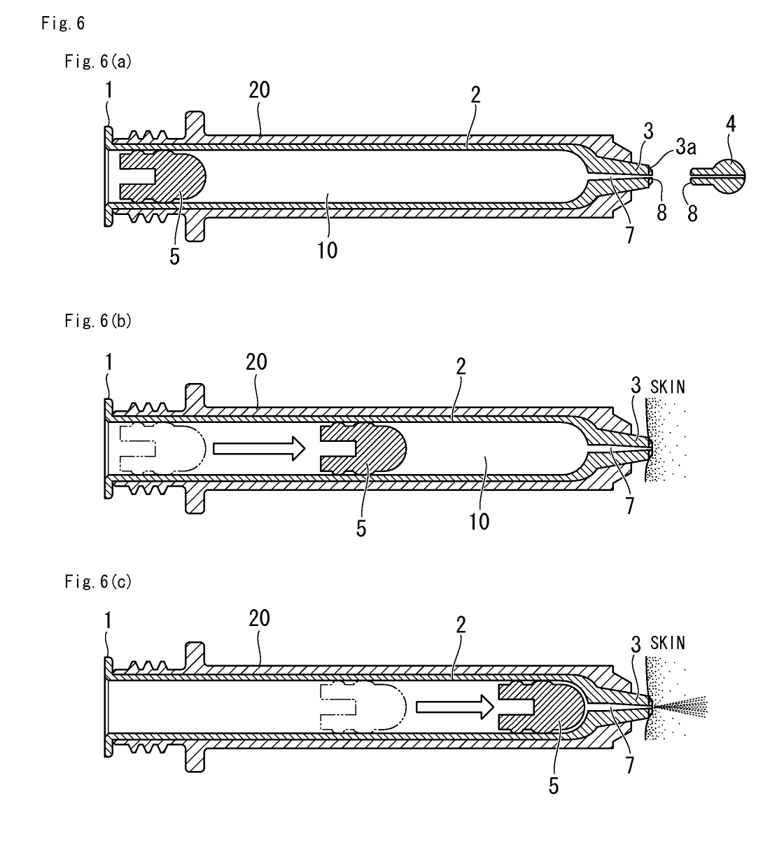

FIG. 6 is a diagram showing a used state of a needleless syringe according to the first embodiment of the present invention.

For the needleless syringe according to the first embodiment of the present invention, the ampoule (1) usable as a syringe is firstly inserted into the holder (20), and then the front edge portion (4) is disconnected at the notched portion (8) by twisting the front edge portion (4) or the like (FIG. 6(a)). It is to be noted that depending on the material of the ampoule (1), the front edge portion (4) may be removed by applying a force from a lateral direction to the front edge portion to break the notched portion (8).

Then, the needleless syringe is applied to the skin at a portion for the drug administration so that the liquid infusing hole (7) becomes cohesive thereto. The cohesiveness with the skin can be increased by the taper portion (3a). The taper portion (3a) is formed in a state projected from the front edge of the holder (20), so that the holder (20) does not touch the skin, and the hygiene is maintained.

Then, the movable stopper (5) is pushed out (FIG. 6 (b)) by the plunger (24) or the like shown in FIG. 7. The drug solution (10) pushed by the movable stopper (5) is emitted through the liquid infusing hole (7) from the infusing opening to be administered within the skin. At this time, since the liquid infusing hole (7) has its diameter gradually made smaller from the ampoule body (2) to the side of the front edge portion (4), it is made possible to reduce the infusing pressure loss of the drug solution (10) and to make the bore diameter at the time of injection small.

Then, the movable stopper (5) pushed out by the plunger (24) or the like reaches the bottom face portion (2a) of the ampoule body (2), thereby emitting the entire amount of the drug solution (10) (FIG. 6(c)). At this time, the shock absorber provided at the bottom face portion (2a) cushions the impact of the movable stopper (5), thereby reducing the load on the bottom face portion (2a) and suppressing the sound of collision between the movable stopper (5) and the bottom face portion (2a) of the ampoule body (2). As a shock absorber, gelled materials whose major ingredient is silicone can be mentioned.

After using the syringe unit, for example, the ampoule (1) usable as a syringe may be taken out from the holder (20) to be disposed. Since the ampoule (1) usable as a syringe basically has no syringe needle, infection due to the used syringe needle will not occur. Also, since there is no need to separate the needle and the ampoule, use of a resin ampoule enables a thermal disposal as is.

When the needleless syringe is used for the next time, another ampoule (1) usable as a syringe may be inserted into the holder (20).

Thus, when the drug solution (10) of 0.5 cc is actually administered, the injection time is as short as 0.15-0.25 sec., and the injection pressure is 54.92 MPa in an initial stage, 12.75-18.63 MPa in an intermediate stage, and 3.43 MPa in a final stage, so that an injection time and an injection pressure suitable for the needleless injection can be achieved. It is to be noted that for a plunger used for the injection, a known plunger may be used including a particle-gun type having helium gas or the like stored therein that is injected by a gas pressure or one that is injected by a momentum (extrusion force) given by a spring or the like, regardless of the embodiment.

It is to be noted that while in this embodiment, a use as a needleless syringe is taken as an example for the description, the syringe unit according to this embodiment can be used as the syringe utilizing a syringe needle. FIG. 7 is a diagram showing a usage state of the syringe unit according to this embodiment in case a syringe needle is utilized.

As shown in FIG. 7, by connecting a syringe needle (23) having a syringe needle connecting portion (22) to the syringe unit (the ampoule (1) usable as a syringe and the holder (20)) according to the present invention, and by using a plunger (24), a use as a normal syringe utilizing the syringe needle is made possible. For this purpose, the ampoule (1) according to this embodiment has a shape where a syringe needle can be attached to the liquid infusing portion (3) after having removed the above-mentioned front edge portion (4). In this case, the drug solution (10) within the ampoule (1) may be pushed out by the plunger (24). Also, in order to push out the drug solution (10) by the movable stopper (5) within the ampoule (1), the movable stopper (5) may be pressed by the front edge of the plunger (24) as shown in FIG. 7.

Also, when thus used, an empty ampoule (1) may be used by measuring the drug solution (10) out of a vial container or the like. In this case, the drug solution (10) can be suctioned into the ampoule (1) by the plunger (24), or the front edge of the plunger (24) may be engaged with the movable stopper (5) as shown in FIG. 7 in order to suction the drug solution (10) with the movable stopper (5) within the ampoule (1). Specifically, an engaging convex portion (24a) of the plunger (24) and the engaging concave portion (5a) of the movable stopper (5) are engaged, and by pulling the movable stopper (5), the drug solution (10) can be suctioned, and the ampoule (1) can be filled with the drug solution (10).

It is to be noted that a method of connecting the syringe needle (23) to the syringe unit is not limited as long as the syringe needle (23) can be fixed. Also, the shape of the plunger (24) for moving the movable stopper (5) may be of any shape, and a method of linking the plunger (24) and the movable stopper (5) may be one that is tolerant of the frictional force between the movable stopper (5) and the internal surface of the ampoule body (2) so that the movable stopper (5) can be moved without fault.

### 4. Manufacturing Process of Ampoule Usable as Syringe

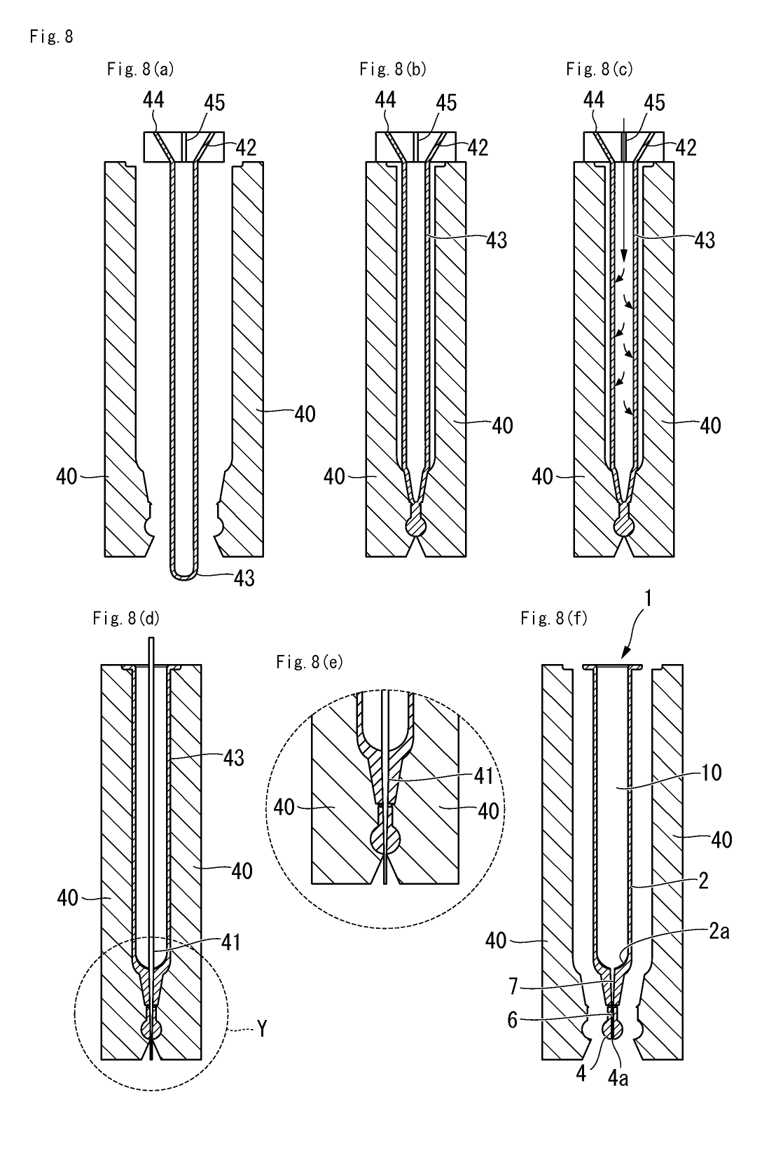

FIG. 8 shows a process explanatory diagram for describing an example of the manufacturing process of the needleless syringe ampoule (1) according to the present invention.

In FIG. 8, a molding die is composed of a pair of main-molding dies (40) and a sub-molding die (41). The main-molding dies (40) form the ampoule body (2), the liquid infusing portion (3), and the front edge portion (4) of the needleless syringe ampoule (1), and the sub-molding die (41) forms the through-hole (6) including the liquid infusing hole (7).

Firstly, the pair of the main-molding dies (40) is set in a state separated from left to right facing each other in a molding position, and moved to the lower side (in a direction where the pair of the main-molding dies (40) mutually approach) of a die (42) for blow molding. The die (42) has a function of pushing out a parison (43) heated to the molding temperature from a push-out opening (44), and a function of infusing pressurized gas such as compressed air from an intake hole (45).

Then, from the push-out opening (44) of the die (42) to between the main-molding dies (40), the parison (43) that is a hollow thermoplastic material semi-fused is pushed out at a low speed (FIG. 8(a)).

When the parison (43) is pushed out for an appropriate length, the main-molding dies (40) are united so that the parison (43) is cut at the bottom portion of the main-molding dies (40) (FIG. 8(b)), and then a compressed air is blown from the intake hole (45) of the die (42) into the sack-like parison (43) to be inflated along the surface of the main-molding die (40), so as to form the ampoule body (2), the liquid infusing portion (3), and the front edge portion (4) of the ampoule (1) usable as a syringe (FIG. 8(c)).

Then the die (42) is taken out, and before the parison (43) cools down, the sub-molding die (41) is inserted to the center of the liquid infusing portion (3) and the front edge portion (4) in order to form the through-hole (6) including the liquid infusing hole (7) (FIG. 8(d)). FIG. 8 (e) is an enlarged view of a portion Y in FIG. 8(d).

When the through-hole (6) shown in FIG. 8(e) is formed, the sub-molding die (41) having a minimum diameter of 0.15-0.17 mm is provided penetrating at a portion corresponding to the liquid infusing portion (3) and the front edge portion (4), to form a coaxial through-hole (6). Since it is difficult to form a small hole (liquid infusing hole) (7) of 0.15-0.17 mm in diameter by welding, such a sub-molding die (41) is provided, so that by removing the sub-molding die (41) at a required timing, the small liquid infusing hole (7) of 0.15-0.17 mm in diameter can be formed.

Lastly, the main-molding dies (40) and the sub-molding die (41) are pulled away, and by sealing one end (4a) of the front edge portion (4) opened by the sub-molding die (41), the ampoule (1) usable as a syringe according to the first embodiment of the present invention is completed (FIG. 8(f)).

### II SECOND AND THIRD EMBODIMENTS

Hereinafter, the second and third embodiments will be described based on the drawings.

### 1. Ampoule Usable as Syringe

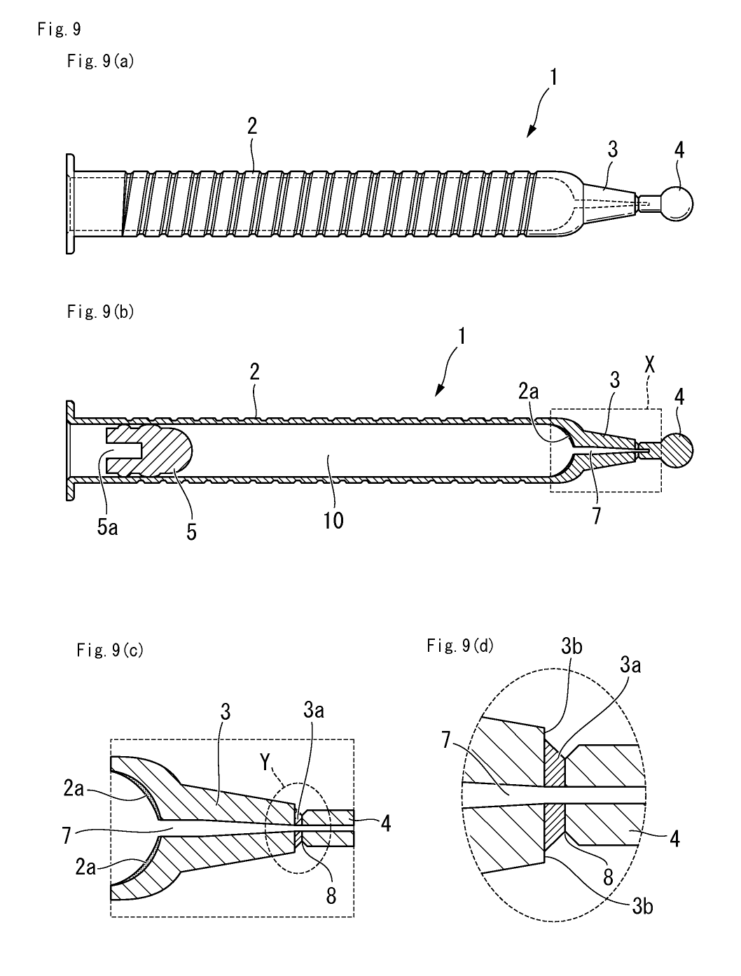

FIG. 9 is a diagram showing an ampoule (1) usable as a syringe according to the second embodiment of the present invention. FIG. 9(a) is a side view of the ampoule (1) usable as a syringe (also referred to as a syringe ampoule), FIG. 9(b) is a cross-sectional view thereof, FIG. 9(c) is an enlarged view of a portion X in FIG. 9(b), and FIG. 9(d) is an enlarged view of a portion Y in FIG. 9(c). Hereinafter, taking a needleless syringe as an example of the ampoule (1) usable as a syringe, a needleless syringe ampoule, an ampoule body (2), a liquid infusing portion (3), a front edge portion (4), and the like will be described.

In FIG. 9, the ampoule (1) usable as a syringe according to the embodiment of the present invention is composed of a front edge portion (4) which is removed upon use; an ampoule body (2) which can accommodate a drug solution (10); and a liquid infusing portion (3) connecting the front edge portion (4) and the ampoule body (2); the front edge portion (4), the ampoule body (2), and the liquid infusing portion (3) are integrally formed; a through-hole (6) is formed in the front edge portion (4), the ampoule body (2), and the liquid infusing portion (3) penetrating from the ampoule body (2) to the midway through the front edge portion (4); the front edge portion (4) has a sealing portion (4a) sealing the through-hole (6); and the ampoule body (2) is able to place a movable stopper (5) in contact with an internal surface of the ampoule body (2), and to be filled with the drug solution (10) hermetically-sealed in the through-hole (6) between the movable stopper (5) and the sealing portion (4a); wherein a spiral concavity and convexity are formed on an external surface of the ampoule body (2).

Preferably, the ampoule body (2) is formed approximately like a hollow cylindrical object. Moreover, the ampoule body (2) has a spiral concave and convex of a 2 mm pitch, for example.

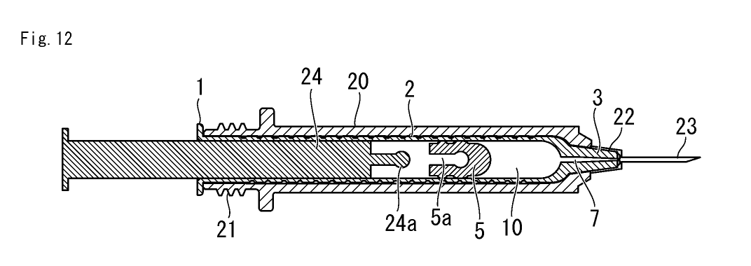

The movable stopper (5) inserted in the ampoule body (2) preferably has a concavity and a convexity formed on a surrounding side face thereof. The front edge (head) of the movable stopper (5) is preferably formed a convex curve of the same shape as that of the bottom face (2a) of the ampoule body (2). Also, at a back end of the movable stopper (5), an engaging portion for engaging with a plunger (24) or the like shown in FIG. 12 for enabling the move of the movable stopper (5) is formed. For example, as shown in FIG. 9(b), an engaging concave portion (5a) is formed.

The concavity and the convexity on the surrounding side face of the movable stopper (5) are formed in order to contact with the inner surface of the ampoule body (5) and to hermetically seal the drug solution (10) between the movable stopper (5) and the sealing portion (4a). Therefore, the airtightness with the ampoule body (2) is increased by the movable stopper (5), so that the drug solution (10) can be hermetically-sealed reliably, and the friction between the movable stopper (5) and the internal surface of the ampoule body (2) is reduced by the concavity and the convexity of the movable stopper, thereby preventing the situation where the movement speed of the movable stopper (5) is reduced due to the injection by the needleless injection. Thus, the decrease in injection pressure of the drug solution (10) can be suppressed.

To describe in more detail, while the concavity and the convexity formed on the surrounding side face of the movable stopper (5) contacts with the internal surface of the ampoule body (2), the contact area of the movable stopper (5) and the internal surface of the ampoule body (2) can be made smaller by the concavity and the convexity compared to the case where the side face of the movable stopper (5) is formed flat. Accordingly, the friction between the movable stopper (5) and the internal surface of the ampoule body (2) is reduced as described above. Also, while only a single tier of the concavity and the convexity of the movable stopper (5) may provide hermetically-sealed state with the inner ampoule body (2), multiple tiers can steadily maintain the hermetically-sealed state with other tiers of the concavity and the convexity even if a contact of a single tier is weak.

The liquid infusing hole (7) is formed in the liquid infusing portion (3) and the liquid infusing hole (7) extends from the hollow portion of the ampoule body (2) to the midway through the front edge portion (4). The liquid infusing hole (7) preferably has its diameter decreased as the diameter approaches the front edge portion (4) from the ampoule body (2) to the front edge portion (4). More specifically, the diameter of the liquid infusing hole (7) on the side of the front edge portion (4) formed smaller than the diameter of the liquid infusing hole (7) on the side of the ampoule body (2). The former diameter is formed about 0.15-0.17 mm and the latter diameter is formed about 0.6 mm.

It is to be noted that the shape of the front edge portion (4) is not limited to that shown in FIG. 9, and another shape may be used. Also, the liquid infusing hole (7) may penetrate to any position of the midway in the front edge portion (4).

While one liquid infusing hole (7) is formed in the second embodiment of the present invention, a plurality of liquid infusing holes may be formed as the liquid infusing hole (7). Examples of the liquid infusing holes can be mentioned as shown in FIG. 2.

Also, the taper portion (3a) tapering off from the liquid infusing portion (3) to a side of the front edge portion (4) is formed at the front edge of the liquid infusing portion (3) on the side of the front edge portion (4). Moreover, a contacting portion (3b) is provided around the taper portion (3a) for contacting the front edge of the liquid infusing portion (3) to the skin more closely, and a notched portion (8) is provided between the liquid infusing portion (3) and the front edge portion (4).

The notched portion (8), which is for disconnecting the front edge portion (4) from the liquid infusing portion (3), is a notch (e.g. a circular cord) formed by being dented inward along the diameter of the ampoule (1), for example, Accordingly, when a force is applied to the front edge portion (4) by a twist of the front edge portion (4) or the like, the stress concentrates on the notched portion (8). Therefore, the front edge portion (4) and the liquid infusing portion (3) can be easily disconnected on the boundary of the notched portion (8).

For example, the taper portion (3a) is formed about 0.3 mm in height and about 2.0-1.2 mm in width (diameter), and the flat contacting portion (3b) is formed around the taper portion (3a), which increase the cohesiveness of the liquid infusing portion (3) with the skin. Also, it is made possible to make the drug solution (10) reach within the skin more reliably without causing dripping at the time of the needleless injection.

To describe in more detail, the size of the taper portion (3a) along the central axis of the liquid infusing portion (3) is preferably shorter than a sunken depth of the front end of the liquid infusing portion (3) compared to the state before pressing the liquid infusing portion (3) to the skin when the front edge of the liquid infusing portion (3) is pressed to the skin to be contacted closely. Therefore, not only the taper portion (3a) but also the flat contacting portion (3b) of the liquid infusing portion (3) can contact to the skin closely. Thus, it is made possible to increase the area of the liquid infusing portion (3) contacted closely to the skin compared to the case where the contacting portion (3b) is not provided.

It is to be noted that while the taper portion (3a) is formed in the second embodiment of the present invention, the taper portion (3a) need not be formed as long as the high level of cohesiveness with the skin can be maintained.

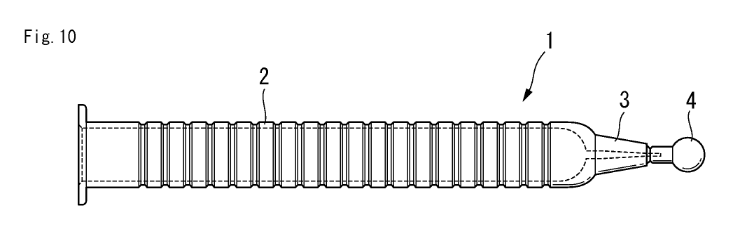

FIG. 10 is a side view of the ampoule (1) usable as a syringe according to another embodiment (the third embodiment) of the present invention.

In FIG. 10, the ampoule (1) usable as a syringe according to this embodiment has a plurality of annular concavities and convexities formed on an external surface. By forming the concavities and the convexities on the external surface of the ampoule (1) usable as a syringe, the strength of the ampoule (2) can be increased.

### 2. Usage

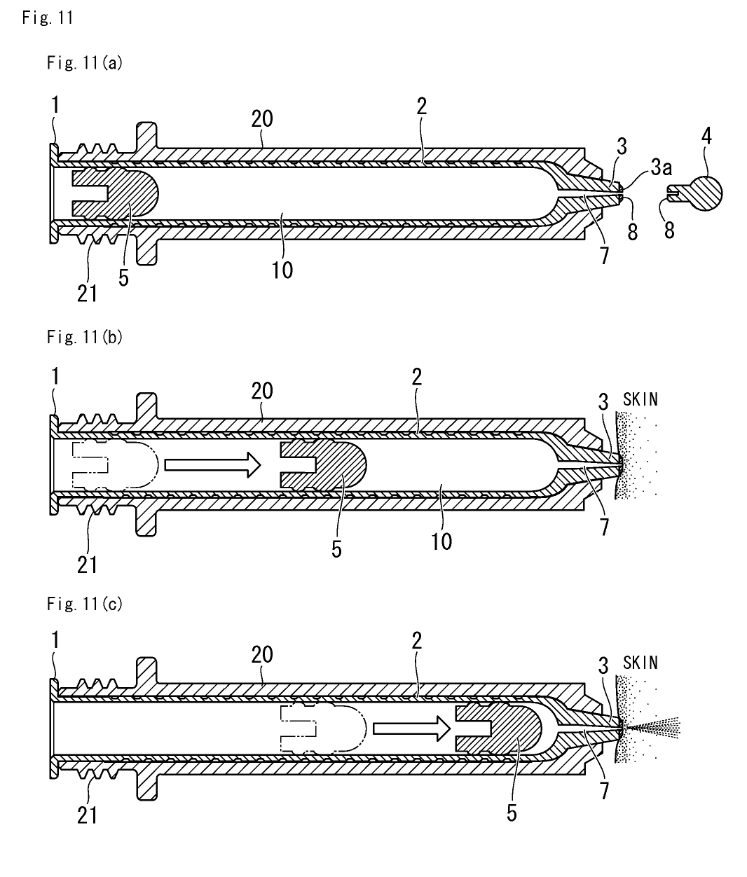

FIG. 11 is a diagram showing a usage state of a needleless syringe according to the second (and the third) embodiment of the present invention.

For the needleless syringe according to the second (and the third) embodiment of the present invention, the ampoule (1) usable as a syringe is firstly inserted into the holder (20). The holder (20) is formed as having approximately the same inner diameter as an outer diameter of the ampoule (1). The holder (20) is shaped so that the ampoule (1) can just fit at a location where the front edge of the liquid infusing portion (3) is slightly out from the holder (20). Also, a connecting portion (21) for connecting to a plunger or the like is formed in the holder (20).

When the ampoule (1) is inserted, such a holder (20) accommodates the ampoule body (2) and the liquid infusing portion (3) to hold the ampoule (1).

Then, the front edge portion (4) is cut off from the notched portion (8) by twisting the front edge portion (4) or the like (FIG. 11(a)). If the ampoule formed of a cyclic olefin copolymer is used, the front edge portion (4) can be cut off more easily, and the front edge of the liquid infusing portion (3) has its surface smoothly formed, so that the cohesiveness of the front edge of the cutoff liquid infusing portion (3) with the skin can be improved.

Then, the needleless syringe is applied to the skin at a portion for the drug administration so that the liquid infusing hole (7) becomes cohesive thereto. The cohesiveness with the skin can be increased by the taper portion (3a) and the contacting portion (3b). The taper portion (3a) is formed in a state projected from the front edge of the holder (20), so that the holder (20) does not touch the skin, and the hygiene is maintained.

Then, the movable stopper (5) is pushed out (FIG. 11 (b)) by the plunger (24) or the like shown in FIG. 12. The drug solution (10) pushed by the movable stopper (5) is emitted through the liquid infusing hole (7) from the infusing opening to be administered within the skin. At this time, since the liquid infusing hole (7) has its diameter made gradually smaller from the ampoule body (2) to the side of the front edge portion (4), it is made possible to reduce the infusing pressure loss of the drug solution (10) and to make the bore diameter at the time of injection small.

Then, the movable stopper (5) pushed out by the plunger (24) or the like reaches the bottom face portion (2a) of the ampoule body (2), thereby emitting the entire amount of the drug solution (FIG. 11(c)). At this time, the shock absorber provided at the bottom face portion (2a) cushions the impact of the movable stopper (5), thereby reducing the load on the bottom face portion (2a) and suppressing the sound of collision of the movable stopper (5) and the bottom face portion (2a) of the ampoule body (2). As a shock absorber, gelled materials whose major ingredient is silicone can be mentioned.

It is to be noted that while this embodiment has been described taking the use using the holder (20) as an example, a plunger or the like can be directly connected to the ampoule (1) usable as a syringe without using the holder (20). When the ampoule (1) is used by directly connecting the plunger or the like, the handling becomes easier since a task of inserting the ampoule (1) into the holder (20) becomes unnecessary.

It is to be noted that while in this embodiment, a use as a needleless syringe is taken as an example for the description, the syringe unit can be used as the syringe utilizing a syringe needle. FIG. 12 shows a usage state of the syringe unit according to this embodiment in case a syringe needle is utilized.

As shown in FIG. 12, by connecting a syringe needle (23) having a syringe needle connecting portion (22) to the syringe unit (the ampoule (1) and the holder (20)) according to the present invention, and by using the plunger (24), a use as a normal syringe utilizing the syringe needle is made possible. In this case, the drug solution (10) within the ampoule (1) may be pushed out by the plunger (24), or in order to push out the drug solution by the movable stopper (5) within the ampoule (1), the movable stopper (5) may be pressed by the front edge of the plunger (24) as shown in FIG. 12.

Also, when thus used, an empty ampoule (1) may be used by measuring the drug solution (10) out of a vial container or the like. In this case, the drug solution (10) can be suctioned into the ampoule (1) by the plunger (24), or the front edge of the plunger (24) may be engaged with the movable stopper (5) as shown in FIG. 12 in order to suction the drug solution (10) with the movable stopper (5) within the ampoule (1). Specifically, an engaging convex portion (24a) of the plunger (24) and an engaging concave portion (5a) of the movable stopper (5) are engaged, and by pulling the movable stopper (5), the drug solution (10) can be suctioned, and the ampoule (1) can be filled with the drug solution (10).

It is to be noted that a method of connecting the syringe needle (23) to the syringe unit is not limited as long as the syringe needle (23) can be fixed. Also, the shape of the plunger (24) for moving the movable stopper (5) may be of any shape, and a method of linking the plunger (24) and the movable stopper (5) may be one that is tolerant of the frictional force between the movable stopper (5) and the internal surface of the ampoule body (2) so that the movable stopper (5) can be moved without fault.

### INDUSTRIAL APPLICABILITY

The ampoule usable as a syringe and the ampoule unit usable as a syringe according to the present invention are beneficial as being easy to handle, and being able to perform a drug administration which is excellent in quantitative accuracy and in hygienic aspect.

1-14. (canceled)

15. An ampoule usable as a syringe for needleless syringe comprising:
a front edge portion (4) which is removed when the ampoule is used;
an ampoule body (2) which can accommodate a drug solution (10); and
a liquid infusing portion (3) connecting the front edge portion (4) and the ampoule body (2);
wherein the front edge portion (4), the ampoule body (2), and the liquid infusing portion (3) being integrally formed;
a through-hole (6) being formed in the front edge portion (4), the ampoule body (2), and the liquid infusing portion (3) penetrating from the ampoule body (2) to the midway of the front edge portion (4);
the front edge portion (4) having a sealing portion (4a) sealing the through-hole (6); and
the ampoule body (2) being able to place a movable stopper (5) in contact with an internal surface of the ampoule body (2), and being able to accommodate the drug solution (10) hermetically-sealed in the through-hole (6) between the movable stopper (5) and the sealing portion (4a).

16. The ampoule (1) usable as a syringe as claimed in claim 15,
wherein a liquid infusing hole (7), that is a portion of the through-hole (6) formed in the liquid infusing portion (3), has a diameter decreased as the diameter approaches from the ampoule body (2) to the front edge portion (4), wherein the diameter of the liquid infusing hole (7) at the front edge portion (4) is from 0.15 mm to 0.17 mm.

17. The ampoule (1) usable as a syringe as claimed in claim 16, wherein a taper portion (3a) tapering off from the liquid infusing portion (3) to a side of the front edge portion (4) is formed at a tip of the liquid infusing portion (3) on the side of the front edge portion (4).

18. The ampoule (1) usable as a syringe as claimed in claim 16,
wherein a taper portion (3a) tapering off from the liquid infusing portion (3) to a side of the front edge portion (4) is formed at a tip of the liquid infusing portion (3) on the side of the front edge portion (4), and
a contacting portion (3b) having a flat surface is provided around the taper portion (3a).

19. The ampoule (1) usable as a syringe as claimed in claim 16,
wherein a portion in the front edge portion (4) connected to the liquid infusing portion (3) is provided with a notched portion (8) for cutting off the front edge portion (4) from the liquid infusing portion (3).

20. The ampoule (1) usable as a syringe as claimed in claim 16,
wherein at a bottom face portion (2a) provided on a side of the liquid infusing portion (3) in the internal surface, the ampoule body (2) has a shock absorb means for absorbing an impact of the movable stopper (5).

21. The ampoule (1) usable as a syringe as claimed in claim 16,
wherein a spiral concavity and convexity are formed on an external surface of the ampoule body (2).

22. The ampoule (1) usable as a syringe as claimed in claim 16,
wherein a plurality of annular concavities and convexities are formed on an external surface of the ampoule body (2).

23. The ampoule (1) usable as a syringe as claimed in claim 16,
wherein the ampoule body (2) accommodates the drug solution (10).

24. The ampoule (1) usable as a syringe as claimed in claim 16,
further comprising a movable stopper (5) in a hollow portion of the ampoule body (2).

25. The ampoule (1) usable as a syringe as claimed in claim 16,
further comprising a movable stopper (5) in a hollow portion of the ampoule body (2), wherein a concavity and a convexity contacting with an internal surface of the ampoule body (2) are formed on a side face of the movable stopper (5).

26. The ampoule (1) usable as a syringe as claimed in claim 16,
further comprising a movable stopper (5) in a hollow portion of the ampoule body (2), wherein a drug solution (10) is accommodated in the through-hole (6) hermetically sealed by the movable stopper (5).

27. An ampoule usable as a syringe for needleless syringe comprising:
a front edge portion (4) which is removed when the ampoule is used;
an ampoule body (2) which can accommodate a drug solution (10); and
a liquid infusing portion (3) connecting the front edge portion (4) and the ampoule body (2);
wherein the front edge portion (4), the ampoule body (2), and the liquid infusing portion (3) being integrally formed;
a through-hole (6) being formed in the front edge portion (4), the ampoule body (2), and the liquid infusing portion (3) penetrating from the ampoule body (2) to the midway of the front edge portion (4);
the front edge portion (4) having a sealing portion (4a) sealing the through-hole (6); and
the ampoule body (2) being able to place a movable stopper (5) in contact with an internal surface of the ampoule body (2), and being able to accommodate the drug solution (10) hermetically-sealed in the through-hole (6) between the movable stopper (5) and the sealing portion (4a),
wherein at a bottom face portion (2a) provided on a side of the liquid infusing portion (3) in the internal surface, the ampoule body (2) has a shock absorb means for absorbing an impact of the movable stopper (5).

28. An ampoule usable as a syringe for needleless syringe comprising:
a front edge portion (4) which is removed when the ampoule is used;
an ampoule body (2) which can accommodate a drug solution (10); and
a liquid infusing portion (3) connecting the front edge portion (4) and the ampoule body (2);
wherein the front edge portion (4), the ampoule body (2), and the liquid infusing portion (3) being integrally formed;
a through-hole (6) being formed in the front edge portion (4), the ampoule body (2), and the liquid infusing portion (3) penetrating from the ampoule body (2) to the midway of the front edge portion (4);
the front edge portion (4) having a sealing portion (4a) sealing the through-hole (6); and
the ampoule body (2) being able to place a movable stopper (5) in contact with an internal surface of the ampoule body (2), and being able to accommodate the drug solution (10) hermetically-sealed in the through-hole (6) between the movable stopper (5) and the sealing portion (4a),
wherein a spiral concavity and convexity are formed on an external surface of the ampoule body (2).

29. An ampoule usable as a syringe for needleless syringe comprising:
a front edge portion (4) which is removed when the ampoule is used;
an ampoule body (2) which can accommodate a drug solution (10); and
a liquid infusing portion (3) connecting the front edge portion (4) and the ampoule body (2);
wherein the front edge portion (4), the ampoule body (2), and the liquid infusing portion (3) being integrally formed;
a through-hole (6) being formed in the front edge portion (4), the ampoule body (2), and the liquid infusing portion (3) penetrating from the ampoule body (2) to the midway of the front edge portion (4);
the front edge portion (4) having a sealing portion (4a) sealing the through-hole (6); and
the ampoule body (2) being able to place a movable stopper (5) in contact with an internal surface of the ampoule body (2), and being able to accommodate the drug solution (10) hermetically-sealed in the through-hole (6) between the movable stopper (5) and the sealing portion (4a),
wherein a plurality of annular concavities and convexities are formed on an external surface of the ampoule body (2).

30. A syringe unit for needleless syringe comprising:
an ampoule for needleless syringe (1);
a movable stopper (5) accommodated in the ampoule (1); and
a holder (20) accommodating the ampoule (1);
wherein the ampoule (1) comprises:
a front edge portion (4) which is removed upon use;
an ampoule body (2) which can accommodate a drug solution (10); and a liquid infusing portion (3) connecting the front edge portion (4) and the ampoule body (2);
the front edge portion (4), the ampoule body (2), and the liquid infusing portion (3) being integrally formed,
a through-hole (6) being formed in the front edge portion (4), the ampoule body (2), and the liquid infusing portion (3) penetrating from the ampoule body (2) to the midway through the front edge portion (4);
the front edge portion (4) having a sealing portion (4a) sealing the through-hole (6), and
the ampoule body (2) being able to place a movable stopper (5) in contact with an internal surface of the ampoule body (2), and being able to accommodate the drug solution (10) hermetically-sealed in the through-hole (6) between the movable stopper (5) and the sealing portion (4a).

31. The syringe unit as claimed in claim 30, wherein the ampoule (1) and the holder (20) are integrally formed.
